(19) United States
(12) Patent Application Publication
(10) Pub. No.: US 2009/0171417 A1
(43) Pub. Date: Jul. 2, 2009

(54) SYSTEM AND METHODS FOR EMG-TRIGGERED NEUROMUSCULAR ELECTRICAL STIMULATION

(76) Inventor: Benjamin J. Philipson, Hashmonaim (IL)

Correspondence Address: DANIEL J SWIRSKY, 55 REUVEN ST., BEIT SHEMESH, 99544, IL

(21) Appl. No.: 11/968,255

(22) Filed: Jan. 2, 2008

(51) Int. Cl. A61N 1/36

(52) U.S. Cl. 607/48

(57) ABSTRACT

A system and methods for EMG-triggered NMES include a display of a target reference signal indicator and an EMG signal indicator. The target reference signal may be automatically adjusted in a continuous manner over time until the detected EMG signal reaches the target reference signal. Once the EMG signal reaches the target reference signal, a stimulation signal is sent to stimulate the muscle or muscles to contract. The stimulation signal includes an exponentially increasing portion so as to minimize pain. The entire system may be controlled via a single control knob.

### FIELD AND BACKGROUND OF THE INVENTION

The present invention is directed to a system and methods for EMG-triggered neuromuscular electrical stimulation (NMES).

EMG-triggered NMES is a known method for treatment and improvement of muscle function for stroke and spinal cord injury patients. Often, following a stroke, control signals from the brain cannot reach some muscles. The low level of electrical activity in these muscles prevents them from contracting, resulting in increasingly weaker muscles. EMG-triggered NMES is a method wherein even a very slight electro-myographical (EMG) signal from an attempt to activate a muscle may be detected and amplified. After detection of EMG signals, a stimulation signal is sent to the muscle or muscles, causing them to contract. This results in a biofeedback loop wherein each contraction strengthens the muscle, resulting in greater EMG signals. Eventually, the goal is to re-teach the muscles to voluntarily contract on their own, without further need for external stimulation signals.

An example of a system for EMG-triggered NMES is disclosed in U.S. Publication Number 2005/0113883 to Sandgaard et al. The system disclosed therein includes a reference signal which can be raised or lowered by discrete amounts depending on the registered signal for an attempt to activate the muscle.

There remains a need for improved systems and methods, which are easy to operate at home by the patient, without the need for a special caregiver, while minimizing pain and maximizing the biofeedback effect.

### SUMMARY OF THE INVENTION

In accordance with embodiments of the present invention, there is provided a system for muscle rehabilitation including a set of electrodes for placement against skin in a vicinity of a muscle or muscles to be rehabilitated, wherein the set of electrodes is configured to detect an EMG signal or signals upon contraction of the muscle and is further configured to stimulate the muscle or muscles, a processor in electrical communication with the electrodes, the processor including a receiver for receiving the detected EMG signal from the set of electrodes, a comparator for comparing an EMG signal parameter from the received EMG signal to a reference signal parameter, an adjustor for adjusting the reference signal based on the received EMG signal, wherein the adjusting is done in a continuously increasing or continuously decreasing manner, and a stimulator for providing a stimulation signal to the set of electrodes when the received EMG signal parameter reaches the reference signal parameter. The system further includes a display with a target reference signal indicator representing a target reference signal at which the stimulation signal is provided and adjustable based on the received EMG signal, and an EMG signal indicator representing a signal level of the received EMG signal during an activation attempt of the muscle or muscles, wherein the EMG signal indicator can be visually compared to the target reference signal indicator.

According to additional aspects of the present invention, there is provided a method for rehabilitating a muscle of a subject. The method includes providing a target reference signal to the subject, detecting an EMG signal during an activation attempt, the activation attempt being an attempt by the subject to contract the muscle, comparing the detected EMG signal to the target reference signal, if the detected EMG signal is lower than the target reference signal, lowering the target reference signal in a continuous manner over time until the detected EMG signal reaches the target reference signal, and when the detected signal reaches the target reference signal, stimulating the muscle of the subject to contract.

According to yet additional aspects of the present invention, there is provided a method for rehabilitating a muscle or muscles of a subject. The method includes providing a target reference signal to a subject, detecting a signal for an activation attempt by the subject, comparing the detected signal to the target reference signal, when the detected signal reaches the target reference signal, stimulating the muscle or muscles of the subject to contract. The stimulating is done with a stimulation signal having an exponentially increasing portion and a stimulation portion, wherein the stimulation portion is at a level sufficient to contract the muscle or muscles, and wherein the increasing portion is between zero and the level sufficient to contract the muscle.

According to yet additional aspects of the present invention, there is provided a system for muscle rehabilitation. The system includes a set of electrodes for placement against skin in a vicinity of a muscle or muscles to be rehabilitated and configured to detect an EMG signal upon an attempted contraction of the muscle or muscles, and further configured to provide a stimulation signal to stimulate the muscle or muscles to contract, and a control box, including a processor in electrical communication with the set of electrodes and a control knob positioned on the control box, the control knob configured for an initial setup mode of the system for setup of a level of the stimulation signal.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of various embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration of a system for muscle rehabilitation in accordance with embodiments of the present invention;

FIG. 2 is a block diagram illustration of a processor located within the system of FIG. 1, showing the various components included therein;

FIG. 3 is a flow-chart illustration of a method of EMG-triggered NMES, in accordance with embodiments of the present invention;

FIG. 4 is a graphical illustration of a cycle of EMG-triggered NMES, including an initial set-up phase, followed by a repeatable sequence including an activation attempt, a stimulation phase and a resting phase;

FIG. 5 is a graphical illustration of a continuous reference signal in comparison with a detected EMG signal, with examples of reference signal adjustment;

FIG. 6 is a view of a display of the system of FIG. 1, in accordance with embodiments of the present invention; and

FIGS. 7A-7C are graphical illustrations of stimulation signals used during the stimulation phase of the cycle shown in FIG. 4.

It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION OF THE INVENTION

In the following detailed description, numerous specific details are set forth in order to provide a thorough understanding of the present invention. It will be understood by those of ordinary skill in the art that the present invention may be practiced without these specific details. In other instances, well-known methods, procedures, components and structures may not have been described in detail so as not to obscure the present invention.

The present invention relates to a system and method for EMG-triggered neuromuscular electrical stimulation (NMES), wherein a subject is prompted to produce a muscle signal, and wherein when the muscle signal reaches a reference signal, the muscle is in turn stimulated to contract, thereby providing a feedback mechanism for rehabilitating the muscle and nerve pathways associated with contraction of the muscle.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

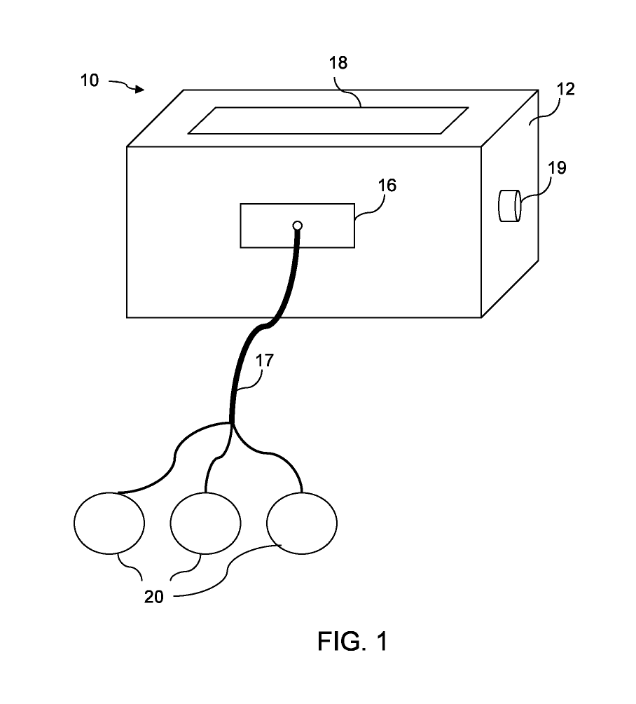

Reference is now made to FIG. 1, which is a schematic illustration of a system 10 for muscle rehabilitation. System 10 includes a control/display box 12, a set of electrodes 20 attached to control/display box 12 via a cable 17 to an input/output interface 16, a processor 14 located within control/display box 12, a display 18 located on control/display box 12, and a control knob 19 positioned on control/display box 12. Electrodes 20 are configured to be placed against the skin in a vicinity of a muscle or muscles to be rehabilitated, and are used for both detection and stimulation of muscle signals. The type and number of electrodes 20 may vary, as is known in the art. For example, electrodes such as CurveZ reusable electrodes manufactured by Selective Med Components Inc. or Electrotherapy electrodes manufactured by Lead-Lok Inc. or any other manufacturer of similar electrodes may be used. In some embodiments, 3-5 electrodes are used. In some embodiments, individual electrodes are used separately for EMG detection and for stimulation, and in other embodiments, the same electrodes are used for EMG detection and stimulation. Moreover, a single muscle may be detected and/or stimulated or multiple muscles may be detected and/or stimulated simultaneously. The invention is not limited to these numbers of electrodes, and any suitable number of electrodes may be used.

Upon an attempt to activate the muscle, electro-myographic (EMG) signals are detected by electrodes 20 and are sent to processor 14 via input/output interface 16. A reference signal is stored within processor 14. If the reference signal is reached by the EMG signals, as determined within processor 14, a stimulation signal is sent from processor 14 to electrodes 20 via input/output interface 16, forming a closed-loop biofeedback system. The reference signal may be variable or adjustable, as described further hereinbelow. Control/display box 12 is a portable box, having dimensions of, but not limited to, ˜10.5″×7″×5″ so that it can be used at home or even during travel. Display 18 displays a representation of the reference signal as well as a representation of the EMG signals obtained during an activation attempt, as will be described in greater detail hereinbelow. Control knob 19 controls an on/off function and an initial setup configuration for stimulation signal level.

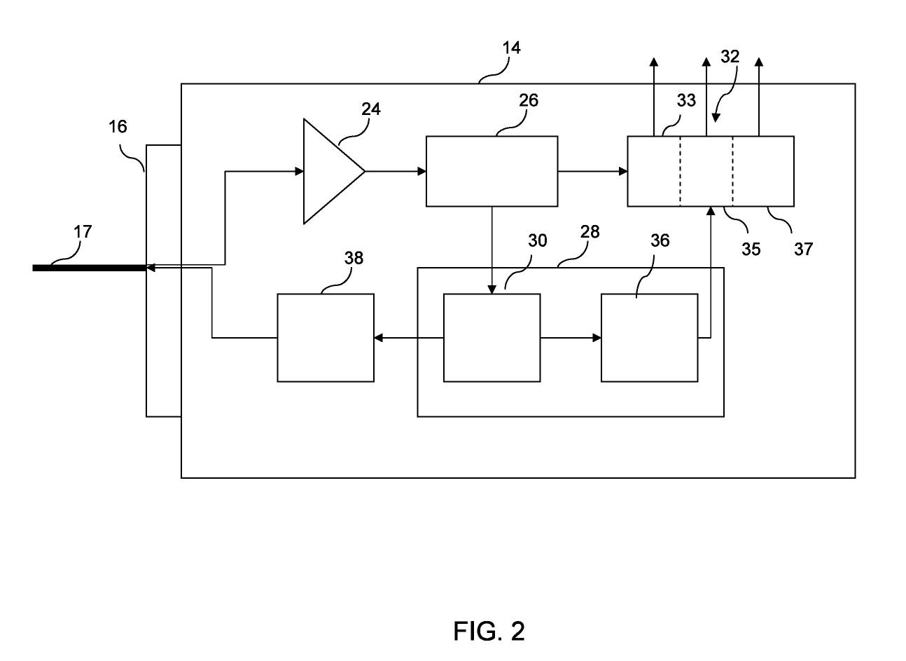

Reference is now made to FIG. 2, which is a block diagram illustration of processor 14, showing the various components included therein. Processor 14 has an input/output interface 16 for receiving EMG signals and for sending stimulation signals to/from electrodes via cable 17. Processor 14 has an amplifier 24 and a filter 26 for processing EMG signals received via input/output interface 16. Amplifier 24 may include one or multiple amplifiers, and may be, for example, INA326 manufactured by Texas Instruments or AD8221 manufactured by Analog devices etc. Filter 26 may be any filter known in the art, such as LMV716 manufactured by National Semiconductor or AD8605 manufactured by Analog Devices or OPA335 manufactured by Texas Instruments. Processor 14 further includes a signal processor 28, for receiving amplified, filtered EMG signals, comparing the received signals to a reference signal, and determining whether the reference signal should be adjusted and whether a stimulation signal should be sent to the electrodes based on the received signals. The comparison to the reference signal may include any defined parameter, such as amplitude, magnitude, frequency, frequency pattern, template matching, signal extraction methods, digital signal processing or analog signal processing methods or any other measurable signal parameter or biological signal processing methods or combinations thereof. Signal processor 28 includes a reference signal comparator 30 for determining whether the received EMG signal reached the reference signal, and a reference signal adjustor 36, for adjusting the reference signal based on the received EMG signal. Processor 14 further includes a stimulator 38, for sending a stimulation signal to the electrodes via input/output interface 16. Stimulator 38 is in electrical communication with reference signal comparator 30 of signal processor 28, such that when a determination is made that the received EMG signal reached a reference signal criterion, reference signal comparator 30 sends a command to stimulator 38 to send a stimulation signal. It is a particular feature of the present invention, that the reference signal is a continuously changing parameter, and is adjusted according to measured data based on the latest obtained signal or signals. Processor 14 further includes a display output 32, wherein display output 32 includes an EMG signal display portion 33, a reference signal display portion 35, and an audio component 37. EMG signal display portion 33 is for receiving the processed EMG signal and sending a representation of the EMG signal to display 18. Reference signal display portion 35 is for receiving either the adjusted or non-adjusted reference signal from reference signal adjustor 36, and sending a representation of the reference signal to display 18. Both EMG signal display portion 33 and reference signal display portion 35 are activated during an EMG activation attempt phase. After the reference signal criterion has been reached, EMG signal display portion 33 is disabled and reference signal display portion 35 is “frozen”. Audio component 37 is for sending an audio signal such as music, to an audio amplifier during a relaxation phase and may also be used to provide an audio prompt for prompting of a new activation attempt. Details of the phases will be described in greater detail hereinbelow.

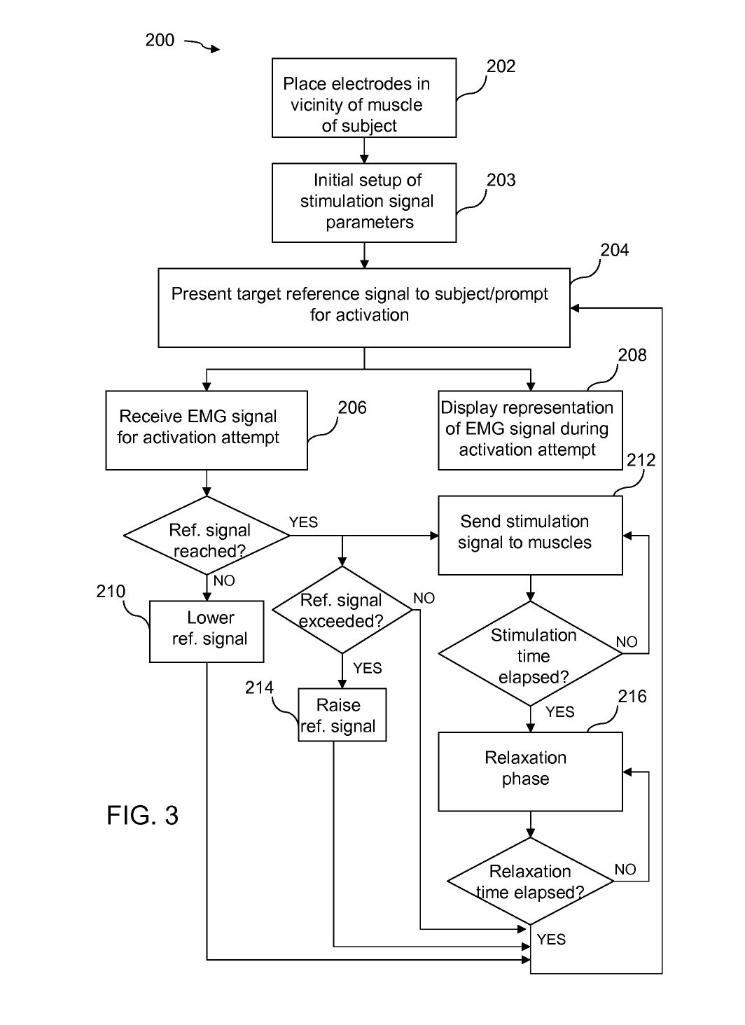

Reference is now made to FIG. 3, which is a flow-chart illustration of a method 200 of EMG-triggered NMES, in accordance with embodiments of the present invention. First, electrodes are placed (step 202) on the subject in the vicinity of the muscle or muscles to be rehabilitated. An initial setup configuration is provided (step 203) to determine the required strength of the stimulation signal. A target reference signal is displayed (step 204) to the subject on display 18, and the subject is prompted by a visual and/or audio signal to activate the muscle. In some embodiments, the display of the target reference signal is the prompting, while in other embodiments, additional prompting may be done, such as verbally prompting the subject, for example. When the subject attempts to activate the muscle, an EMG signal is received (step 206) by processor 14. Simultaneously, the EMG signal obtained during the activation attempt is representatively displayed (step 208) on display 18, so that the subject can view the EMG signal as compared to the target reference signal. If the indicated reference signal level is not reached, reference signal adjustor 36 lowers (step 210) the reference signal, and the new reference signal is presented (step 204) to the subject while prompting for activation continues. If the reference signal is reached, stimulator 38 sends (step 212) a stimulation signal to the muscle for a predetermined period of time. In some embodiments, the stimulation signal lasts between 2 and 24 seconds, and in further embodiments, the stimulation signal lasts approximately 5 or 6 seconds. When the stimulation time is completed, audio component 37 sends an audio signal to signify the relaxation phase. The audio signal may be a soothing audio signal, such as music, to enhance relaxation. The relaxation phase continues (step 216) for a predetermined period of time. In some embodiments, the relaxation phase lasts between 2 and 24 seconds, and in further embodiments, the relaxation phase lasts approximately 10-14 seconds. When the relaxation phase is complete, the subject is prompted (step 204) to activate the muscle again, either by stopping the relaxing audio signal and/or by presenting a new audio signal, for example. The target reference signal is adjusted according to the last EMG signal(s) received, even when the last EMG signal reached the target reference signal. If the prior EMG signal exceeded the reference signal, the reference signal is increased (step 214) and then the increased reference signal is presented (step 204) to the user. If the prior EMG signal was reached, but not exceeded, the reference signal remains the same.

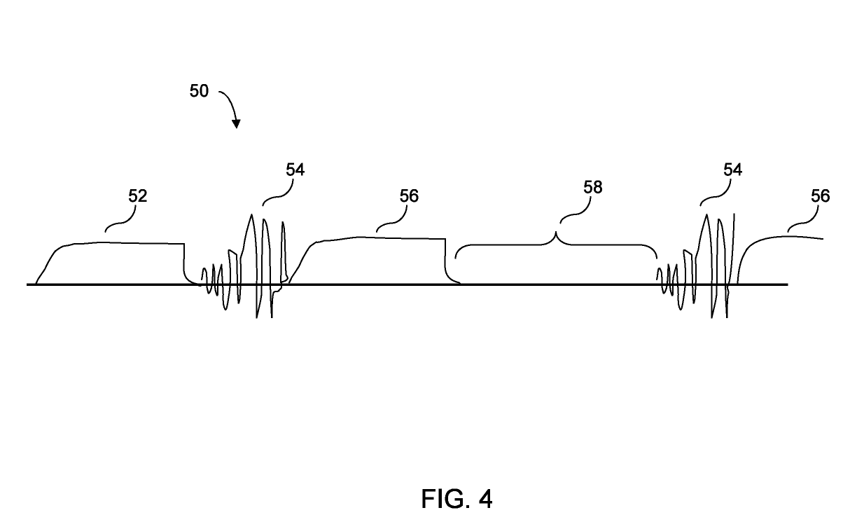

Reference is now made to FIG. 4, which is a graphical illustration of a cycle 50 of EMG-triggered NMES, in accordance with embodiments of the present invention. Cycle 50 includes an initial set-up phase 52, followed by a repeatable sequence including an activation attempt 54, a stimulation phase 56, a resting phase 58 and another activation attempt 54, etc. In one embodiment, the initial set-up phase 52 starts automatically upon turning the device clockwise ‘on’ with the knob and lasts for a limited stimulation time (between 2 and 20 seconds and in some embodiments approximately 10 seconds) which is sufficient time to set the stimulation signal to obtain satisfactory stimulation of the muscle. As described above with reference to FIG. 3, after the set-up phase 52 is complete, the subject is prompted to activate the muscle. Prompting may be done by displaying a target reference signal, by verbally prompting the subject, by providing an audio signal, or by any other method. The subject attempts to activate the muscle, as indicated by activation attempt 54. EMG signals as a result of activation attempt 54 are detected and compared to a reference signal. If the reference signal is reached, the stimulation phase 56 begins. Stimulation phase 56 is followed by a resting phase 58. Resting phase 58 is necessary to allow the muscle to relax before another activation attempt is made. During resting phase 58, music may be played to help the subject relax the muscle and to indicate that during this relaxation period no muscle movement attempts are to be made to avoid tiring of the muscles. During this relaxation period the EMG measuring and the muscle stimulation circuits may be disabled. Once resting phase 58 is complete, the biofeedback cycle begins again with a display of the target reference signal and prompting for an activation attempt, followed by an activation attempt 54, etc.

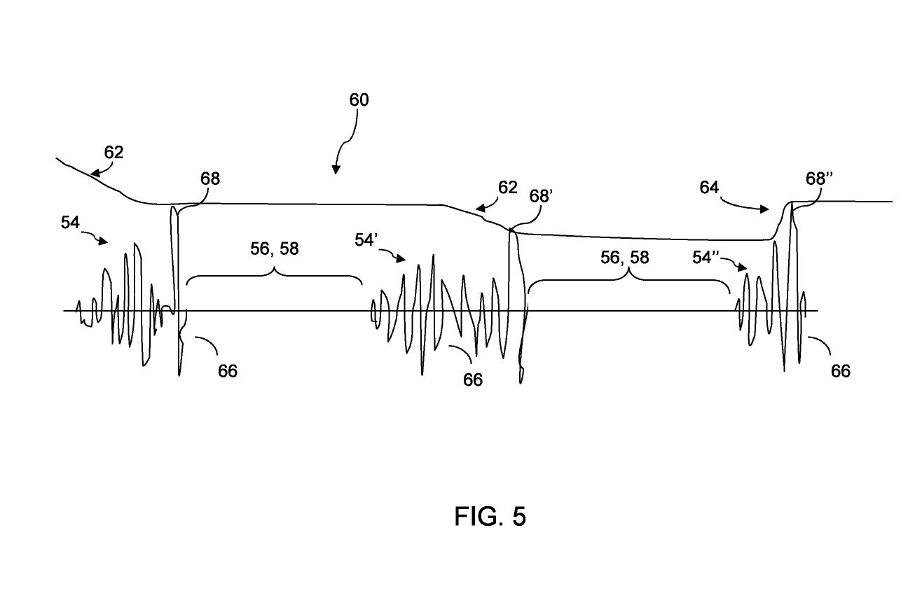

Reference is now made to FIG. 5, which is a graphical illustration of a continuous reference signal in comparison with a detected EMG signal, with examples of reference signal adjustment. A target reference signal 60 is represented as a continuous line, which can increase or decrease in a continuous, rather than a discrete, fashion. Reference signal comparator 30 compares received EMG signals 66 received during activation attempt 54 to target reference signal 60. Target reference signal 60 is variable, and depends on this comparison. Thus, as shown in FIG. 5, wherein in a first activation attempt 54, the peak signal 68 did not reach the initial target reference signal, target reference signal 60 is shown with a decreasing slope 62, which levels out at the level of peak signal 68. The decrease may begin as a “bleeding” or gradual lowering of the reference signal when the reference signal is not reached and continues until the EMG signal reaches the continuously decreasing target reference signal. This gradual lowering may be exponential. Similarly, in the example shown in FIG. 5, in a second activation attempt 54′, peak signal 68′ does not reach the new target reference signal and as such, another decreasing slope 62 is shown on target reference signal 60. A third activation attempt 54″ results in a peak signal 68″ which exceeds the new target reference signal, resulting in an increasing portion 64 on target reference signal 60. Thus, target reference signal 60 is of a continuously changing level, which depends on each individual activation attempt. It should be readily apparent that the sequence of events shown in FIG. 5 is exemplary, and that any change in peak signal will result in a corresponding change in the target reference signal. In this way, the measurement parameters can be made sensitive enough to provide stimulation even for very weak EMG signals, and are continuously adjusted to challenge the subject to increase the EMG signals when possible and to stimulate the subject to put in more effort after a successful attempt. It should be noted that even when the target reference signal increases, it may again decrease so that the subject can reach the target without becoming frustrated and without fatiguing the muscles.

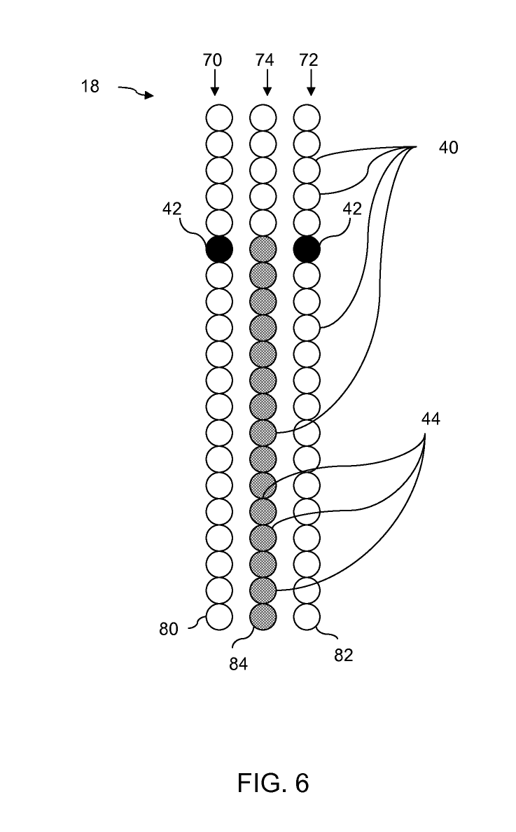

Reference is now made to FIG. 6, which is a view of display 18, in accordance with embodiments of the present invention. Display 18 includes a target reference signal indicator 42, which represents a goal for the subject to attempt to reach, and which varies in accordance with target reference signal 60 as described above with reference to FIG. 5. Displaying the target reference signal provides encouragement and a focus to the subject, which can enable a more successful attempt at activation. Display 18 further includes an EMG signal indicator 44, representing a real-time indication of the EMG signal level during contraction of the muscle. For purposes of the present application, the term “signal level” is understood as a measure of signal achieved in accordance with a pre-defined parameter. Thus, if the parameter is signal amplitude, the “signal level” represents a value of the signal amplitude. Similarly, if the parameter is a different parameter, such as frequency, the “signal level” represents a value of frequency. Similarly, the “signal level” may represent a value of magnitude, frequency, frequency pattern, template matching or any other measurable signal parameter or process. The parameter for the “signal level” corresponds to the parameter of the reference signal used for comparison. By displaying both target reference signal indicator 42 and EMG signal indicator 44, the subject is able to view the attempt and the goal simultaneously, providing immediate encouragement. In some embodiments, target reference signal indicator 42 and EMG signal indicator 44 are LEDs. However, it should be readily apparent that many other types of indicators may be used such as LCD display or others. In some embodiments, target reference signal indicator 42 is a different color or pattern than EMG signal indicator 44. In one embodiment, as shown in FIG. 6, three columns of LEDs are displayed: an outer left-hand column 70, an outer right-hand column 72 and a middle column 74. Outer left-hand column 70 includes a first outer left-hand column LED 80, positioned at the bottom of the column, and representing the lowest signal level for the reference signal, and additional LEDs going up for each increasing signal level. The total number of outer left-hand column LEDs may vary, and may depend on the size of the LEDs and the available space on display 18. Similarly, outer right-hand column 72 includes a first outer right-hand column LED 82, positioned at the bottom of the column, and representing the lowest signal level for the reference signal, and additional LEDs going up for each increasing signal level. The total number of outer right-hand column LEDs may vary, and may depend on the size of the LEDs and the available space on display 18. Moreover, signal levels may be discrete or continuous, and may be determined based on expected performance ranges. Outer left-hand column 70 and outer right-hand column 72 are used for target reference signal indicator 42, wherein target reference signal indicator 42 includes one LED from outer left-hand column 70 at a particular signal level and another LED from outer right-hand column 72 at the same signal level, wherein the signal level is determined by the adjustment method described above with reference to FIGS. 3 and 5. Thus, these two LEDs, positioned at a particular signal level, form a “goal” for the subject to aim for. It should be readily apparent that in some embodiments, a different number of columns may be used for target reference signal indicator 42. For example, a single column may represent target reference signal indicator 42. Alternatively, representation of target reference signal indicator 42 may be done without the use of columns. Middle column 74 also includes a first middle column LED 84, positioned at the bottom of the column, and representing the lowest signal level, and an additional LED for each increasing signal level. The total number of middle column LEDs may vary, and may depend on the size of the LEDs and the available space on display 18. For a given muscle activation, the EMG signal level is displayed by lighting up all of the LEDs from the lowest level until the obtained signal level (bar representation) or by lighting up one LED after the other (dot representation) presenting a ball rolling towards the goal. Thus, in this embodiment, EMG signal indicator 44 includes a column of LEDs. It should be readily apparent that many other configurations are possible as well. For example, a single LED displayed at a single signal level may be used.

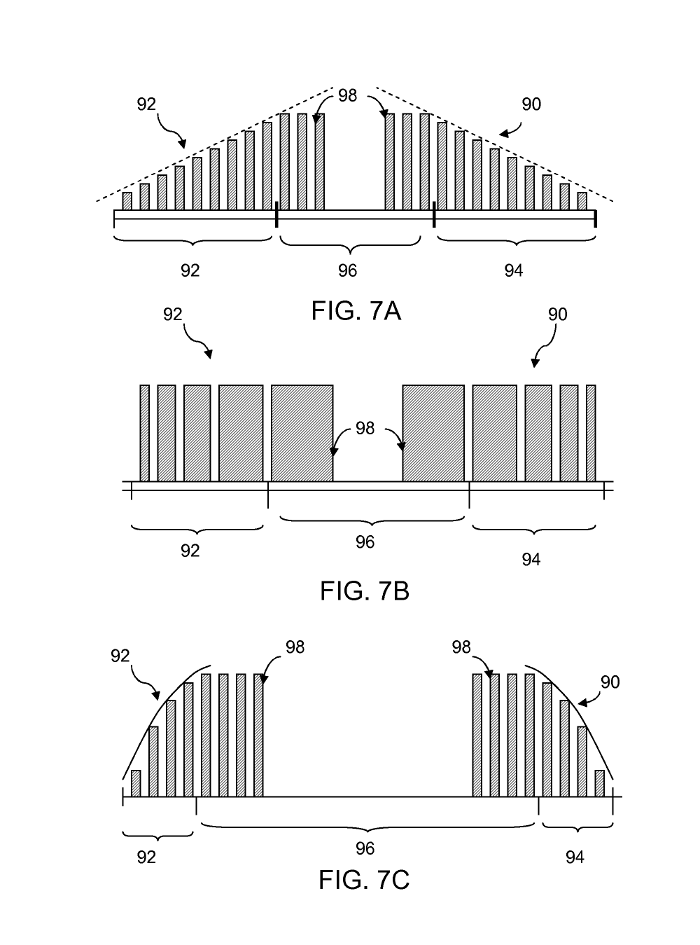

Reference is now made to FIGS. 7A-7B, which are graphical illustrations of known methods of stimulation signals which can be used during a stimulation phase, and to FIG. 7C, which is a graphical illustration of a method of providing a stimulation signal 90 during stimulation phase 56 in accordance with embodiments of the present invention. Stimulation signal 90 is a signal sent by stimulator 38 to electrodes 20, which causes the muscle undergoing rehabilitation to contract. Generally, stimulation signal 90 has an increasing portion 92 and a decreasing portion 94. For example, as shown in FIG. 7A, increasing portion 92 may include a linear increase and a linear decrease of stimulation signal strength. Stimulation signal 90 may be comprised of multiple pulses 98, each of which may be mono- or bi-phasic. Alternatively, as shown in FIG. 7B, increasing portion 92 may include an increase of pulse width and decreasing portion 94 may include a decrease of pulse width. Stimulation signal 90 may be comprised of multiple pulses 98, each of which may be mono- or bi-phasic. However, it has been unexpectedly discovered by the inventor of the present application, that by applying an exponentially ramped up and/or ramped down stimulation signal, pain upon contraction of the muscle may be minimized. As shown in FIG. 7C, increasing portion 92 includes an exponential increase of stimulation signal strength and decreasing portion 94 includes an exponential decrease of stimulation signal strength. Stimulation signal 90 may be comprised of multiple pulses 98, each of which may be mono- or bi-phasic. In addition to reduction of pain, the “reward” for having succeeded at the activation attempt is felt immediately with an exponentially increasing signal, as can be seen by comparing the curve of FIG. 7A to the curve of FIG. 7C. This increases the overall effectiveness of the biofeedback. Thus, there is a distinct advantage in providing a varying stimulation signal to the muscle, wherein the stimulation signal has an exponentially increasing portion 92 and an exponentially decreasing portion 94 and generally also includes a stimulating portion 96. The stimulating portion 96 is the portion of the stimulation signal which is sufficient to contract the muscle. In some embodiments, an increasing portion 92 and a stimulation portion 96 may be used, without a decreasing portion. Alternatively, a decreasing portion 94 and a stimulation portion 96 may be used, without an increasing portion. The timing of each portion may vary and may be, for example, between 0.5 and 10 seconds. Various other embodiments as well as combinations of the various embodiments are possible as well.

The system of the present invention has been designed so that patients can operate system 10 independently, even with compromised motor abilities. Other systems generally include confusing menus with multiple options for changing a variety of parameters such as frequency, pulse width, time on/off, stimulation time, sensitivity, stimulation level, etc. As such, it would be extremely difficult or impossible for an average user to properly adjust the settings. In the system of the present invention, a single key parameter has been isolated from the remaining parameters and is accessible to a patient so that the patient can use the system on his/her own. The single parameter is stimulation signal strength, which must be set for each individual and is generally set for each session. This parameter, stimulation signal strength, may be set using control knob 19, for example, by turning control knob 19 in a clockwise direction until the required stimulation level is reached, as determined by actual muscle activity (i.e., the hand or foot is raised). The remaining parameters and settings may be separately controlled or changed via settings at the back or bottom of or hidden within control/display box 12 or via wireless or other communication means. In some embodiments, control knob 19 is also used for on/off operation. In other embodiments, a separate on/off knob or switch may be used.

A method for EMS-triggered NMES in accordance with embodiments of the present invention may thus include the following steps. Initially, after application of electrodes 20, the subject switches control/display box 12 on by turning control knob 19 in a clockwise direction. The subject continues to turn control knob 19 in a clockwise direction until the required stimulation level is reached, as determined by actual muscle activity (i.e., the hand or foot is raised). Immediately following this stimulation setup phase 52 (as shown diagrammatically in FIG. 4), system 10 switches into an operating mode where the cycle of activation attempt, stimulation and relaxation phase is implemented. The stimulation setup phase is set for a predetermined period of time (approximately 10 seconds). If the subject does not reach the required stimulation level within the predetermined period of time, control knob 19 can be turned off (counter-clockwise, in the present example), and then turned on again to reset the predetermined setup phase to complete the setup. Thus, setup and operation can be done easily with one hand by any patient using system 10.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for muscle rehabilitation, the system comprising:
a set of electrodes for placement against skin in a vicinity of a muscle to be rehabilitated, said set of electrodes configured to detect an EMG signal upon contraction of said muscle, and further configured to stimulate said muscle;
a processor, wherein said set of electrodes is in electrical communication with said processor, said processor comprising:
a receiver for receiving said detected EMG signal from said set of electrodes;
a comparator for comparing an EMG signal parameter from said received EMG signal to a reference signal parameter;
an automatic adjustor for adjusting said reference signal based on said received EMG signal, wherein said adjusting is done in a continuously increasing or continuously decreasing manner; and
a stimulator for providing a stimulation signal to said set of electrodes when said received EMG signal parameter reaches said reference signal parameter; and
a display comprising:
a target reference signal indicator, said target reference signal indicator representing a target reference signal at which said stimulation signal is provided, said target reference signal adjustable based on said received EMG signal; and
an EMG signal indicator representing a signal level of said received EMG signal during an activation attempt of said muscle,
wherein said EMG signal indicator can be visually compared to said target reference signal indicator.

2. The system of claim 1, further comprising a control box, wherein said processor is located in said control box and wherein said display is positioned on said control box.

3. The system of claim 2, further comprising a control knob positioned on said control box, said control knob configured for setting up a level of said stimulation signal.

4. The system of claim 1, said display further comprising an audio component.

5. The system of claim 4, wherein said audio component is configured to provide music during a relaxation phase.

6. The system of claim 1, wherein said target reference signal indicator and said EMG signal indicator are LEDs.

7. The system of claim 6, wherein said target reference signal indicator LED and said EMG signal indicator LED are different colors.

8. The system of claim 6, wherein said target threshold indicator includes at least one LED at a target threshold signal level, and wherein said EMG signal indicator LED includes multiple LEDs representing varying signal levels during an activation attempt.

9. The system of claim 1, wherein said EMG signal parameter is a signal level.

10. The system of claim 1, wherein said stimulation signal comprises an exponentially increasing portion and a stimulation portion.

11. The system of claim 10, wherein said stimulation signal further comprises an exponentially decreasing portion.

12. A method for rehabilitating a muscle of a subject, the method comprising:
providing a target reference signal to the subject;
detecting an EMG signal during an activation attempt, said activation attempt being an attempt by the subject to contract the muscle;
comparing the detected EMG signal to the target reference signal;
if the detected EMG signal is lower than the target reference signal, lowering the target reference signal in a continuous manner over time until the detected EMG signal reaches the target reference signal; and
when the detected signal reaches the target reference signal, stimulating the muscle of the subject to contract.

13. The method of claim 12, further comprising: if the detected signal is higher than the target reference signal, raising the target reference signal for the next activation attempt.

14. The method of claim 12, further comprising: after said stimulating, initiating a relaxation phase.

15. The method of claim 14, wherein said initiating is done with music.

16. The method of claim 14, further comprising: after said relaxation phase, providing a new target reference signal to the subject, said new target reference signal based on said detected EMG signal.

17. The method of claim 12, wherein said lowering of the target reference signal is done exponentially.

18. The method of claim 12, wherein said providing a target reference signal is done via a display with a target reference signal indicator.

19. The method of claim 18, wherein said display further comprises an EMG signal indicator, and wherein said detecting an EMG signal comprises displaying said EMG signal with said EMG signal indicator.

20. A method for rehabilitating a muscle of a subject, the method comprising:
providing a target reference signal to a subject;
detecting a signal for an activation attempt by the subject;
comparing the detected signal to the target reference signal;
when the detected signal reaches the target reference signal, stimulating the muscle of the subject to contract, said stimulating comprising stimulating with a stimulation signal having an exponentially increasing portion and a stimulation portion, wherein said stimulation portion is at a level sufficient to contract the muscle, and wherein said increasing portion is between zero and said level sufficient to contract the muscle.

21. The method of claim 20, further comprising an exponentially decreasing portion between said level sufficient to contract the muscle and zero.

22. The method of claim 20, wherein said level of said stimulation portion is set via a control knob on a system for EMG-triggered NMES, said control knob also configured for on/off operation of said system.

23. A system for muscle rehabilitation, the system comprising:
a set of electrodes for placement against skin in a vicinity of a muscle to be rehabilitated, said set of electrodes configured to detect an EMG signal upon an attempted contraction of said muscle, and further configured to provide a stimulation signal to stimulate said muscle to contract; and
a control box, comprising:
a processor in electrical communication with said set of electrodes;
a control knob positioned on said control box, said control knob configured for an initial setup mode of said system, said initial setup mode comprising setup of a level of said stimulation signal.
